(19) United States
(12) Patent Application Publication
(10) Pub. No.: US 2013/0023569 A1
(43) Pub. Date: Jan. 24, 2013

(54) Use of Deferiprone for Treatment and Prevention of Iron-Related Eye Disorders

(71) Applicants: Michael Spino, Pickering (CA); Joshua Lawrence Dunaief, Philadelphia, PA (US)

(72) Inventors: Michael Spino, Pickering (CA); Joshua Lawrence Dunaief, Philadelphia, PA (US)

(73) Assignees: THE TRUSTEES OF THE UNIVERSITY OF PENNSYLVANIA, Philadelphia, PA (US); APOTEX TECHNOLOGIES INC., Toronto, ON (CA)

(21) Appl. No.: 13/138,263

(22) PCT Filed: Nov. 12, 2009
(86) PCT No.: PCT/CA2009/001639

§ 371 Date: Oct. 1, 2012

Related U.S. Application Data
(62) Division of application No. 61/147,245, filed on Jan. 26, 2009.

(51) Int. Cl. A61K 31/4422; A61P 25/02; A61P 27/02; A61P 9/10; A61P 27/06; A61P 27/12

(52) U.S. Cl. 514/348

(57) ABSTRACT

There is provided use of orally available or topically applied deferiprone for prevention of iron-induced eye damage. The use may be for preparation of a medicament or in a method of preventing iron-induced eye damage to an eye of a subject at risk for iron-induced eye damage, the method comprising administering a prophylactically effective amount of deferiprone to the subject. There is also provided the use of deferiprone for treatment of iron-related eye disorders. The use may be for preparation of a medicament or in a method of treatment of damage to an eye of a having eye damage associated with iron, the method comprising topically administering a therapeutically effective amount of deferiprone to the subject.

### TECHNICAL FIELD

This invention relates to the treatment of iron-related eye disorders and prophylaxis of iron-related eye disorders. More particularly, this invention relates to use of deferiprone for the treatment and prophylaxis of eye damage associated with iron and/or a metabolic mishandling of iron in the eye.

### BACKGROUND

CA 2,642,778 describes a therapeutically effective amount of deferiprone or deferasirox or physiologically acceptable salts thereof for the prevention, stabilization, treatment, or reversal of iron-induced FRDA disease in patients resulting from mitochondrial iron-induced damage to preferentially reduce the iron stores in the mitochondria. Also for the treatment of other conditions affecting the brain where a key element in the generation of the resultant pathology is the intracellular mishandling of iron. CA 2,642,778 further describes that in yet another embodiment the condition being treated is macular degeneration.

U.S. 2008/0279913 describes a method for treating age-related macular degeneration, blindness or glaucoma using an iron-chelator salicylaldehyde isonicotinoyl hydrazone (SIH). Furthermore, U.S. 2008/0279913 describes a method of treating oxidative stress of the retina in a subject, comprising contacting the retina with an effective amount of a metal chelator, wherein said chelator is SIH, pyridoxal isonicotinoyl hydrazone (PIH), N-(2-hydroxybenzyl)-L-serine (HB-Ser), desferrioxamine (DF) or combinations thereof.

WO 2007/118276 describes treatment and prophylaxis of retinal degenerative diseases. More particularly, WO 2007/118276 contemplates a method for preventing, reducing the risk of development of, or otherwise treating or ameliorating the symptoms of, age-related macular degeneration (AMD) or related retinal conditions in mammals and in particular humans. WO 2007/118276 further provides therapeutic compositions enabling dose-dependent or dose-specific administration of agents useful in the treatment and prophylaxis of age-related macular degeneration or related retinal degenerative conditions.

### SUMMARY

This invention is based, in part, on the understanding that deferiprone, whether administered topically or orally can prevent damage to an eye of a subject at risk for developing iron-induced damage to the eye by providing orally available or topically applied deferiprone to the subject. In some instances, iron-induced damage in the eye may already have occurred and further damage can be prevented using deferiprone.

This invention is also based, in part, on the understanding that deferiprone, when administered topically to the eye, can treat iron-related eye disorders without side effects that are sometimes associated with oral administration of deferiprone.

Iron-related eye disorders and/or iron-induced damage may occur from biochemical mishandling of iron such as might occur due to a deficiency of cellular iron transporters for exporting iron out of the cell, or due to an inadequacy of iron binding proteins resulting in increased labile iron that leads to the production of reactive oxygen species. The appearance of labile iron may also be the result of bleeding at a microscopic or macroscopic level into the eye or its component tissues. Biochemical and/or physical mechanisms may lead to iron-induced damage. Deferiprone, administered orally or topically, can treat the condition and/or prevent the iron-induced damage by interfering with the mechanism of iron-induced toxicity in such ocular disorders.

In illustrative embodiments of the present invention there is provided use of deferiprone for treatment of an iron-related eye disorder selected from the group consisting of: glaucoma, cataract, diabetic retinopathy, hereditary retinal degeneration, retinal detachment, ischemic retinopathy caused by retinal vein or artery occlusions, ischemic optic neuropathy, optic neuritis, and traumatic optic neuropathy.

In illustrative embodiments of the present invention there is provided use of deferiprone for preparation of a medicament for treatment of an iron-related eye disorder selected from the group consisting of: glaucoma, cataract, diabetic retinopathy, hereditary retinal degeneration, retinal detachment, ischemic retinopathy caused by retinal vein or artery occlusions, ischemic optic neuropathy, optic neuritis, and traumatic optic neuropathy.

In illustrative embodiments of the present invention there is provided a use described herein wherein the deferiprone is suitable for oral administration.

In illustrative embodiments of the present invention there is provided a use described herein wherein the deferiprone is suitable for topical administration.

In illustrative embodiments of the present invention there is provided a method of treatment for an iron-related eye disorder selected from the group consisting of: glaucoma, cataract, diabetic retinopathy, hereditary retinal degeneration, retinal detachment, ischemic retinopathy caused by retinal vein or artery occlusions, ischemic optic neuropathy, optic neuritis, and traumatic optic neuropathy, the method comprising administering a therapeutically effective amount of deferiprone to the eye of a subject having an iron-related eye disorder.

In illustrative embodiments of the present invention there is provided a method described herein wherein the deferiprone is administered orally.

In illustrative embodiments of the present invention there is provided a method described herein wherein the deferiprone is administered topically.

In illustrative embodiments of the present invention there is provided use of deferiprone suitable for topical administration for treatment of iron-related eye disorders.

In illustrative embodiments of the present invention there is provided use of deferiprone suitable for topical administration for preparation of a medicament for treatment of iron-related eye disorders.

In illustrative embodiments of the present invention there is provided a use described herein wherein the iron-related eye disorder is selected from the group consisting of age-related macular degeneration, glaucoma, cataract, diabetic retinopathy, hereditary retinal degeneration, retinal detachment, ischemic retinopathy caused by retinal vein or artery occlusions, ischemic optic neuropathy, optic neuritis, and traumatic optic neuropathy.

In illustrative embodiments of the present invention there is provided a use described herein wherein the iron-related eye disorder is age-related macular degeneration.

In illustrative embodiments of the present invention there is provided a use described herein wherein the age-related macular degeneration is exudative.

In illustrative embodiments of the present invention there is provided a use described herein wherein the age-related macular degeneration is nonexudative.

In illustrative embodiments of the present invention there is provided a method of treatment for iron-related eye disorders, the method comprising topically administering a therapeutically effective amount of deferiprone to the eye of a subject having an iron-related eye disorder.

In illustrative embodiments of the present invention there is provided a method described herein wherein the iron-related eye disorder is selected from the group consisting of age-related macular degeneration, glaucoma, cataract, diabetic retinopathy, hereditary retinal degeneration, retinal detachment, ischemic retinopathy caused by retinal vein or artery occlusions, ischemic optic neuropathy, optic neuritis, and traumatic optic neuropathy.

In illustrative embodiments of the present invention there is provided a method described herein wherein the iron-related eye disorder is age-related macular degeneration.

In illustrative embodiments of the present invention there is provided a method described herein wherein the age-related macular degeneration is exudative.

In illustrative embodiments of the present invention there is provided a method described herein wherein the age-related macular degeneration is nonexudative.

In illustrative embodiments of the present invention there is provided a method described herein wherein the subject has previously been treated for an iron-related eye disorder.

In illustrative embodiments of the present invention there is provided a method described herein wherein the subject has previously been treated for age-related macular degeneration.

In illustrative embodiments of the present invention there is provided use of deferiprone for prevention of iron-induced eye damage.

In illustrative embodiments of the present invention there is provided use of deferiprone for preparation of a medicament for prevention of iron-induced eye damage.

In illustrative embodiments of the present invention there is provided a use described herein wherein the iron-induced eye damage is a physical distortion of the retina.

In illustrative embodiments of the present invention there is provided a use described herein wherein the physical distortion of the retina is scarring.

In illustrative embodiments of the present invention there is provided a use described herein wherein the iron-induced eye damage is abnormal vascular growth.

In illustrative embodiments of the present invention there is provided a use described herein wherein the iron-induced eye damage is also associated with an iron-related eye disorder is selected from the group consisting of age-related macular degeneration, glaucoma, cataract, diabetic retinopathy, hereditary retinal degeneration, retinal detachment, ischemic retinopathy caused by retinal vein or artery occlusions, ischemic optic neuropathy, optic neuritis, and traumatic optic neuropathy.

In illustrative embodiments of the present invention there is provided a use described herein wherein the iron-induced eye damage is also associated with age-related macular degeneration.

In illustrative embodiments of the present invention there is provided a use described herein wherein the age-related macular degeneration is exudative.

In illustrative embodiments of the present invention there is provided a use described herein wherein the age-related macular degeneration is nonexudative.

In illustrative embodiments of the present invention there is provided a use described herein wherein the deferiprone is suitable for oral administration.

In illustrative embodiments of the present invention there is provided a use described herein wherein the deferiprone is suitable for topical administration.

In illustrative embodiments of the present invention there is provided a method of preventing iron-induced damage to an eye of a subject at risk for iron-induced eye damage, the method comprising administering a prophylactically effective amount of deferiprone to the subject.

In illustrative embodiments of the present invention there is provided a method described herein wherein the eye damage is a physical distortion of the retina.

In illustrative embodiments of the present invention there is provided a method described herein wherein the physical distortion of the retina is scarring.

In illustrative embodiments of the present invention there is provided a method described herein wherein the eye damage is abnormal vascular growth.

In illustrative embodiments of the present invention there is provided a method described herein wherein the iron-induced eye damage is associated with an iron-related eye disorder is selected from the group consisting of age-related macular degeneration, glaucoma, cataract, diabetic retinopathy, hereditary retinal degeneration, retinal detachment, ischemic retinopathy caused by retinal vein or artery occlusions, ischemic optic neuropathy, optic neuritis, and traumatic optic neuropathy.

In illustrative embodiments of the present invention there is provided a method described herein wherein the iron-induced eye damage is associated with age-related macular degeneration.

In illustrative embodiments of the present invention there is provided a method described herein wherein the age-related macular degeneration is exudative.

In illustrative embodiments of the present invention there is provided a method described herein wherein the age-related macular degeneration is nonexudative.

In illustrative embodiments of the present invention there is provided a method described herein wherein the subject has previously been treated for eye damage associated with iron.

In illustrative embodiments of the present invention there is provided a method described herein wherein the subject has previously been treated for age-related macular degeneration.

In illustrative embodiments of the present invention there is provided a method described herein wherein the deferiprone is administered orally.

In illustrative embodiments of the present invention there is provided a method described herein wherein the deferiprone is administered topically.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

FIG. 1 is two brightfield photomicrographs of 1 μM thick plastic sections of retinas from systemic Cp/Heph double knockout (DKO) mice stained with toluidine blue. The upper panel shows a retina from an untreated mouse age 13 months and the lower panel shows a retina from a 14 month old Cp/Heph knockout treated with oral deferiprone for 5 months.

FIG. 2 is two graphs showing relative quantification of transferrin Receptor (TfR) mRNA levels in the retinas and retinal pigment epithelia (RPE)/choroids of deferiprone treated and untreated wild type mice in retinas (FIG. 2A) and in RPE/choroids (FIG. 2B).

FIG. 3 is a graph illustrating the amount of transferrin receptor messenger ribonucleic acid (TfR mRNA) in the RPE/choroid from a treated eye and an untreated eye. The error bars represent SD of 3 PCR reactions using the same batch of mRNA template.

FIG. 4 illustrates three Perls' stains of 7 and 13 month old untreated DKO mice retinas (FIGS. 4A and 4B, respectively) and of a treated 13 month old DKO mouse (FIG. 4C). The following abbreviations appear in the Figures: RPE, retinal pigment epithelium; ONL, outer nuclear layer; OPL, outer plexiform layer; IPL, inner plexiform layer; GCL, ganglion cell layer. Scale bar: 50 μm.

FIG. 5 is twelve brightfield micrographs of plastic sections of retinas from deferiprone treated and untreated DKO mice and a wild type mouse. FIGS. 5C, 5F and 5I show the results from untreated 12 and 13 month old animals; FIGS. 5A, 5D, 5G and 5J show results from treated age-matched and older DKO mice that received deferiprone 1 mg/ml PO in drinking water for 6-9 months; FIGS. 5B, 5E, 5H and 5K show the pathology found in the retinas of the same animals as 5A, 5D, 5G and 5J, but from an area exhibiting the most severe pathology detected in all sections examined. FIG. 5L is the retina of a wild type mouse showing normal histology at 18 months. The following abbreviations are used in FIGS. 5A-5K: RPE, retinal pigment epithelium; ONL, outer nuclear layer; OPL, outer plexiform layer; IPL, inner plexiform layer; GCL, ganglion cell layer. Scale bar: 50 μm.

FIG. 6 is a graph showing hematocrit values in deferiprone treated and untreated DKO mice.

FIG. 7 is a Kaplan-Meier survival curve for deferiprone treated and untreated DKO mice.

### DETAILED DESCRIPTION

In illustrative embodiments of the present invention, there is provided use of deferiprone suitable for topical administration, such as in eye drops, for treatment of iron-induced eye damage. The use may be for preparation of a medicament. Also provided is a method of treating iron-induced eye damage in an eye of a subject having iron-induced eye damage, the method comprising topically administering a therapeutically effective amount of deferiprone to the subject.

Suitable topical pharmaceutical compositions may be formulated by means known in the art and their mode of administration and dose determined by the skilled practitioner. Many suitable formulations are known, including, polymeric or protein microparticles encapsulating a compound to be released, ointments, pastes, gels, hydrogels, or solutions which can be used topically or locally to administer a compound. Many techniques known to one of skill in the art are described in Remington: the Science & Practice of Pharmacy by Alfonso Gennaro, 20^{th} ed., Lippencott Williams & Wilkins, (2000).

Suitable ophthalmic formulations may be prepared by dissolving deferiprone in water or other ophthalmically suitable carriers. Often carboxymethylcellulose may be included in ophthalmic formulations comprising deferiprone. For example, and without limitation, a suitable topical formulation may include a therapeutically effective or a prophylactically effective amount of deferiprone dissolved in water together with carboxymethyl cellulose 0.5%. Other suitable topical formulations include ophthalmic formulations known to the person of skill in the art.

In clinical practice oral doses from 75 to 100 mg/kg/day have been approved for treating subjects having thalassemia. A 50 kg subject may receive several grams of the drug each day. Deferiprone can induce agranulocytosis in about 1% of thalassemia patients treated with the drug at such doses. The use of deferiprone eye drops alters transferrin receptor concentrations, indicative of a reduction in intracellular iron in the eye. Drops may be administered in a concentration of 1-100 mg/ml of deferiprone three times daily. The half life of deferiprone is only about 2 hours in humans. Repeated dosing using topically administered deferiprone, such as eye drops, does not result in accumulation of the deferiprone. By delivering repeated, smaller doses of deferiprone directly to the eye, the risk of bone marrow suppression and agranulocytosis may be reduced in subjects treated for iron-related eye disorders using topically administered deferiprone compared with orally administered deferiprone. A common adverse effect found in thalassemia patients taking deferiprone orally is nausea and vomiting due to gastrointestinal irritation, which is not a feature of topically applied deferiprone. Thus the topical mode of administration has an advantage of decreasing the risk of causing side effects associated with the use of deferiprone by other administrative routes.

In illustrative embodiments of the present invention, there is provided use of deferiprone suitable for oral administration and/or suitable for topical administration for prevention of iron-induced damage to an eye. The use may be for preparation of a medicament. Also provided is a method of preventing iron-induced eye damage to an eye of a subject at risk for iron-induced eye damage, the method comprising orally administering and/or topically administering a prophylactically effective amount of deferiprone to the subject.

Suitable pharmaceutical compositions may be formulated by means known in the art and their mode of administration and dose determined by the skilled practitioner. For enteral administration, the compound may be administered in a tablet, capsule or dissolved in liquid form. The tablet or capsule may be in an immediate release format or enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. Techniques particular to ophthalmic solutions may be necessary, including pH adjustment to pH 7.4 (range 5.4-8.4) and the relevant use of buffers, osmolarity adjustment to be approximately isotonic; the addition of appropriate bacteriostatic agents; the employment of asceptic and/or sterile techniques and related techniques known to one skilled in the art of formulating topical agents for application to the eye. Many techniques known to one of skill in the art are described in Remington: the Science & Practice of Pharmacy by Alfonso Gennaro, 20^{th} ed., Lippencott Williams & Wilkins, (2000).

Iron-induced eye damage may occur from biochemical mishandling of iron. Non-limiting examples of factors that may lead to mishandling of iron that may lead to iron-induced eye damage include a deficiency of cellular iron transporters for iron, or an inadequacy of iron binding proteins. Iron-induced eye damage can also result from bleeding at a microscopic or a macroscopic level into the eye or its component tissues.

Iron, whether normally occurring or abnormally occurring, that may become involved or may be involved with iron-related eye disorders may be found in any part of the eye, whether intracellular or extracellular, including, but not limited to: retinal tissue, corneal tissue, lens tissue, and other tissues, as well as in various different eye cell types, such as retinal pigment epithelium (RPE) and other eye cells.

Iron-related disorders may involve ocular iron overload and/or deposits, and/or may also involve iron-induced oxidative stress caused by normal iron levels in the eye. Such normal iron levels may be iron that is mishandled. Non-limiting examples of iron-related ocular diseases that involve oxidative stress include macular degeneration, glaucoma, cataract, diabetic retinopathy, hereditary retinal degeneration, retinal detachment, ischemic retinopathy caused by retinal vein or artery occlusions, ischemic optic neuropathy, optic neuritis, and traumatic optic neuropathy. Such occular diseases may be treated using deferiprone.

A subject at risk for developing iron-related damage include, without limitation, subjects at risk for developing one of: macular degeneration, glaucoma, cataract, diabetic retinopathy, hereditary retinal degeneration, retinal detachment, ischemic retinopathy caused by retinal vein or artery occlusions, ischemic optic neuropathy, optic neuritis, and traumatic optic neuropathy. Another non-limiting example of a subject at risk for developing iron-induced eye damage is a subject that has recently had surgery performed on their eye, such as but not limited to, Laser-Assisted In Situ Keratomileusis (LASIK) surgery. Iron may occur within the margin of the ablated zone of such surgeries. Another example of a subject at risk for developing iron-induced eye damage is a subject that has recently had surgery performed, where microvascular hemorrhage accompanies such surgery, including, but not limited to cataract surgery, glaucoma surgery and retinal detachment surgery.

Another example of a subject at risk for developing iron-induced eye damage is a subject diagnosed as having or being at risk for macular degeneration. The subject may be at risk for exudative macular degeneration. The subject may have non-exudative macular degeneration.

Macular degeneration, often called AMD or ARMD (age-related macular degeneration) results in a progressive destruction of the macula. The macula is a part of the eye (and in particular a part of the retina) responsible for sharp, central vision required to read or drive. In AMD, central vision loss may occur due to progressive damage to the macula.

Macular degeneration may be diagnosed as either nonexudative (dry) or exudative (wet). In the exudative form, the growth of new blood vessels occurs in an area, such as the macula, where they are not normally present in healthy subjects. The exudative form of the disease usually leads to more serious vision loss.

Nonexudative AMD is often an early stage of the disease and may result from the aging and thinning of macular tissues, depositing of pigment (often including iron) in the macula or a combination of the two processes. Nonexudative AMD may be diagnosed when yellowish spots known as drusen begin to accumulate from deposits or debris. Often the deposits or debris are from deteriorating tissue. This often occurs around the macula. Gradual central vision loss may occur with dry macular degeneration but is not nearly as severe as exudative AMD symptoms. Nonexudative AMD may progress to a more advanced and damaging form of the eye disease, termed exudative AMD. Subjects having non-exudative AMD may be subjects at risk for developing iron-related eye damage associated with exudative AMD.

In exudative AMD, new blood vessels grow (neovascularization). Exudative AMD occurs with formation of abnormal blood vessels and leakage in the back of the eye. Neovascularization of the abnormal blood vessels may occur beneath the retina and the new blood vessels may leak blood and fluid into the surrounding area. Such leakage may result in deposition of iron. Such leakage may cause permanent damage to an eye. In many cases the leakage damages light-sensitive retinal cells, which die off and create blind spots in central vision. It is thought that it is this sort of activity affects the macula where fine focusing occurs. Neovascularization is an underlying process thought to be involved with exudative AMD and abnormal blood vessel growth. The process creates scarring and often leads to severe central vision loss.

Exudative AMD falls into two categories: classic and occult. In classic exudative AMD, neovascularization and scarring often show very clear, delineated outlines that are observable behind the retina. This type of exudative AMD is sometimes referred to as classic choroidal neovascularization.

In occult exudative AMD, neovascularization behind the retina is not as clear and delineated as it is in classic exudative AMD. Leakage from blood vessels is less evident in occult exudative AMD when compared to classic exudative AMD and typically produces less severe vision loss than classic exudative AMD.

Many forms of macular degeneration are linked to aging and related deterioration of eye tissue crucial for good vision. An association between development of macular degeneration and presence of a variant of a gene known as complement factor H (CFH) has been proposed. Variants of another gene, complement factor B, may also be involved in development of AMD.

Deteriorating, oxygen-starved cells within the retina appear to help trigger neovascularization and accompanying damage in exudative AMD. Neovascularization may be activated by a protein called vascular endothelial growth factor (VEGF). Anti-VEGF drugs have been used to treat exudative AMD.

Risk factors for AMD may include having a family member with AMD, high blood pressure, lighter eye color, obesity, smoking, over exposure to sunlight and high levels of dietary fat. In addition to affecting older populations, AMD occurs more prevalently in females. Macular disease also can result as a side effect of some drugs, such as Aralen (chloroquine, an anti-malarial drug) or phenothiazines. Phenothiazines represent a class of anti-psychotic drugs, including brand names of Thorazine (chlorpromazine, which is also used to treat nausea, vomiting and persistent hiccups), Mellaril (thioridazine), Prolixin (fluphenazine), Trilafon (perphenazine) and Stelazine (trifluoperazine). A subject having any one or more of these risk factors may be a subject at risk for iron-related eye damage. Such a subject may benefit from using deferiprone prophylactically.

Iron is essential for life, primarily because of its role in intermediary metabolism and related activities that involve one-electron redox chemistry in the electron transport chain, and because it serves as a cofactor in heme and iron-sulfer cluster containing proteins. When in excess, or in the absence of factors that maintain control of its transport and storage, iron creates a potentially dangerous electron-transporting system generating oxidative damage through the Fenton reaction. Thus, while biochemical reactions involving iron are needed and necessary, in the absence of the controls that are normally in place, or in the presence of a localized excess of iron that overwhelms the capacity of the system to neutralize the toxic effects of iron, iron reacts with hydrogen peroxide (H202) to produce hydroxyl radical, the most reactive and toxic of the reactive oxygen species (ROS) resulting in oxidative stress that can cause significant damage to membranes, cellular organelles, and even the cells themselves. This oxidative stress may result from mishandled or elevated levels of iron, or may result from increased production of peroxides, which can then interact with the normal cellular iron to produce damaging hydroxyl radicals.

Current evidence suggest that iron may play a critical role in the pathogenesis of age-related macular degeneration (AMD), and it is now known that there are elevated iron levels in the retinas of patients with AMD. Likewise, patients with the rare hereditary disease aceruloplasminemia have iron overload of the brain, retina, and pancreas, leading to degeneration in these organs.

Macular degeneration refers to a family of diseases that are characterized by a progressive loss of central vision associated with abnormalities of Bruch's membrane, the choroid, the neural retina and/or the retinal pigment epithelium and are particularly evident in older subjects, thus the term, age-related macular degeneration, although some forms can be detected as early as the first decade of life. AMD, the most prevalent macular degeneration, is associated with progressive loss of visual acuity in the central portion of the visual field, changes in color vision, and abnormal dark adaptation and sensitivity. Two principal clinical manifestations of AMD are the dry, or atrophic, form, and the wet, or exudative, form. The most significant risk factor for the development of both forms are age and the deposition of drusen, abnormal extracellular deposits, behind the retinal pigment epithelium (RPE). Drusen causes a lateral stretching of the RPE monolayer and physical displacement of the RPE from its immediate vascular supply, leading to the damage that affects the vision.

In addition to macular degeneration, there are several other ocular disorders in which there is iron-induced oxidative stress. Some of these disorders may even have normal levels of iron levels in the eye, but in which iron has been identified as a significant contributor to the consequent pathology. Such ocular diseases as glaucoma, cataracts, diabetic retinopathy, hereditary retinal degeneration, retinal detachment, ischemic retinopathy caused by retinal vein or artery occlusions, ischemic optic neuropathy, optic neuritis, and traumatic optic neuropathy, all of which involve oxidative stress, may be prevented and/or treated by protecting various tissues in the eye from damage that is caused or facilitated by labile iron. Such protection may be provided by using deferiprone. The deferiprone may be administered topically or orally.

While an iron chelator seems like a rational solution to the problem, there are major concerns because none of these patients suffer from generalized iron overload and consequently it may be that in the absence of generalized iron overload, an iron chelator would deplete the body of iron, an essential element for survival. Prior to such fatal effects, it an iron chelator may interfere with intermediary metabolism by reducing the available pool of iron needed by many enzymes in normal everyday biochemical reactions for homeostasis, as well as for the production of red blood cells. Thus the only way to determine whether an agent might be capable of treating and/or preventing the development of iron-related eye disorders is to employ a drug candidate in a living animal that demonstrates suitable pathology and evaluate the effects and whether the toxic effects of a particular iron chelator in the absence of generalized iron overload, would prohibit such use.

In illustrative embodiments of the present invention, there is provided a method described herein wherein the subject has previously been treated for iron-related eye damage.

In illustrative embodiments of the present invention, there is provided a method described herein wherein the subject has previously been treated for age-related macular degeneration.

An “effective amount” of a pharmaceutical composition according to the invention includes a therapeutically effective amount or a prophylactically effective amount. A “therapeutically effective amount” refers to an amount effective, at dosages and for periods of time necessary, to achieve the desired therapeutic result, such as improved pathology of iron-related disorders, including, but not limited to, macular degeneration, glaucoma, cataract, diabetic retinopathy, hereditary retinal degeneration, retinal detachment, ischemic retinopathy caused by retinal vein or artery occlusions, ischemic optic neuropathy, optic neuritis, and traumatic optic neuropathy. A therapeutically effective amount of a compound may vary according to factors such as the mode of administration, disease state, age, sex, and weight of the subject, and the ability of the compound to elicit a desired response in the subject. Dosage regimens may be adjusted to provide the optimum therapeutic response. Often doses of deferiprone suitable for oral administration are from between about 5 mg/kg/day to about 80 mg/kg/day while doses suitable for topical administration are often from about 1 mg/ml to about 100 mg/ml. Examples, without limitation, of suitable doses of deferiprone (where the term “unit” may be replaced with either “ml” or “kg/day”) may include from about 1 mg/unit to about 100 mg/unit; from about 1 mg/unit to about 90 mg/unit; from about 1 mg/unit to about 80 mg/unit; from about 1 mg/unit to about 70 mg/unit; from about 1 mg/unit to about 60 mg/unit; from about 1 mg/unit to about 50 mg/unit; 1 mg/unit to about 40 mg/unit; from about 1 mg/unit to about 30 mg/unit; from about 1 mg/unit to about 20 mg/unit; from about 1 mg/unit to about 15 mg/unit; from about 1 mg/unit to about 14 mg/unit; from about 1 mg/unit to about 13 mg/unit; 1 mg/unit to about 12 mg/unit; from about 1 mg/unit to about 11 mg/unit; from about 1 mg/unit to about 10 mg/unit; from about 1 mg/unit to about 9 mg/unit; from about 1 mg/unit to about 8 mg/unit; from about 1 mg/unit to about 7 mg/unit; from about 1 mg/unit to about 6 mg/unit; from about 1 mg/unit to about 5 mg/unit; from about 1 mg/unit to about 4 mg/unit; from about 1 mg/unit to about 3 mg/unit; from about 1 mg/unit to about 2 mg/unit; from about 2 mg/unit to about 15 mg/unit; from about 2 mg/unit to about 14 mg/unit; from about 2 mg/unit to about 13 mg/unit; 2 mg/unit to about 12 mg/unit; from about 2 mg/unit to about 11 mg/unit; from about 2 mg/unit to about 10 mg/unit; from about 2 mg/unit to about 9 mg/unit; from about 2 mg/unit to about 8 mg/unit; from about 2 mg/unit to about 7 mg/unit; from about 2 mg/unit to about 6 mg/unit; from about 2 mg/unit to about 5 mg/unit; from about 2 mg/unit to about 4 mg/unit; from about 2 mg/unit to about 3 mg/unit; from about 3 mg/unit to about 15 mg/unit; from about 3 mg/unit to about 14 mg/unit; from about 3 mg/unit to about 13 mg/unit; 3 mg/unit to about 12 mg/unit; from about 3 mg/unit to about 11 mg/unit; from about 3 mg/unit to about 10 mg/unit; from about 3 mg/unit to about 9 mg/unit; from about 3 mg/unit to about 8 mg/unit; from about 3 mg/unit to about 7 mg/unit; from about 3 mg/unit to about 6 mg/unit; from about 3 mg/unit to about 5 mg/unit; from about 3 mg/unit to about 4 mg/unit; from about 4 mg/unit to about 15 mg/unit; from about 4 mg/unit to about 14 mg/unit; from about 4 mg/unit to about 13 mg/unit; 4 mg/unit to about 12 mg/unit; from about 4 mg/unit to about 11 mg/unit; from about 4 mg/unit to about 10 mg/unit; from about 4 mg/unit to about 9 mg/unit; from about 4 mg/unit to about 8 mg/unit; from about 4 mg/unit to about 7 mg/unit; from about 4 mg/unit to about 6 mg/unit; from about 4 mg/unit to about 5 mg/unit; from about 5 mg/unit to about 15 mg/unit; from about 5 mg/unit to about 14 mg/unit; from about 5 mg/unit to about 13 mg/unit; 5 mg/unit to about 12 mg/unit; from about 5 mg/unit to about 11 mg/unit; from about 5 mg/unit to about 10 mg/unit; from about 5 mg/unit to about 9 mg/unit; from about 5 mg/unit to about 8 mg/unit; from about 5 mg/unit to about 7 mg/unit; from about and from 5 mg/unit to about 6 mg/ml. A therapeutically effective amount is also one in which any toxic or detrimental effects of the compound are outweighed by the therapeutically beneficial effects.

A “prophylactically effective amount” of a pharmaceutical composition (e.g. a composition comprising deferiprone) according to the invention refers to an amount effective, at dosages and for periods of time necessary to achieve the desired prophylactic result, such as reduced or a lack of iron-induced eye damage. Typically, a prophylactic dose is used in subjects prior to or at an earlier stage of a disease or disorder, so that a prophylactically effective amount may be less than a therapeutically effective amount. Nevertheless, a prophylactically effective amount may be the same or similar to a therapeutically effective amount. The exemplified ranges of therapeutically effective doses may be considered to be suitable exemplified ranges for prophylactically effective amounts.

It is to be noted that dosage values may vary with the severity of the condition to be prevented. For any particular subject, specific dosage regimens may be adjusted over time according to the individual need and the professional judgement of the person administering or supervising the administration of the effective amount. Dosage ranges set forth herein are exemplary only and do not limit the dosage ranges that may be selected by medical practitioners. The amount of active compound(s) in the composition may vary according to factors such as the mode of administration, disease state, age, sex, and weight of the subject. Dosage regimens may be adjusted to provide the optimum prophylactic and/or therapeutic response. For example, a single bolus may be administered, several divided doses may be administered over time or the dose may be proportionally reduced or increased as indicated by the exigencies of the situation.

As used herein, a “subject” may be a human, non-human primate, rat, mouse, cow, horse, pig, sheep, goat, dog, cat, etc. The subject may have, be suspected of having or at risk for having a disease or disorder that often results in iron-induced damage or iron catalyzed oxidative stress even when ocular iron levels are normal (e.g. AMD, dry eye, corneal degeneration or ulcer, glaucoma, cataract, diabetic retinopathy, retinal detachment, hereditary retinal degeneration, eye surgery and other factors discussed herein). Diagnostic methods for various iron-related disorders and the clinical delineation of iron-related disorder diagnoses are known to those of ordinary skill in the art. Those subjects having such disorders or exhibiting iron-induced damage may be suitable for treatment with deferiprone. Those subjects exhibiting or having risk factors for such disorders may be suitable for prophylactic use of deferiprone.

### EXAMPLES

The following examples are illustrative of some of the embodiments of the invention described herein. These examples should not be considered to limit the spirit or scope of the invention in any way.

In the following examples, double knockout (DKO) mice were used where the mice were genetically modified to generate a deficiency in the ferroxidase ceruloplasmin (Cp) and its homologue hephaestin (Heph). These mice develop age-related retinal degeneration due to retinal iron accumulation, as do humans. Since the knockout affects all tissues and organs, they also develop other symptoms of localized iron excess, with a simultaneous inability to adequately utilize the available iron for normal homeostasis, such as making new hemoglobin (they have low hematocrits). These animals also develop neurological deficits, including ataxia and die at an early age, typically 6-9 months, although some may survive to 12 or 13 months with significant impaired ability to function The retinas of these Cp/Heph deficient mice accumulate iron in the RPE and photoreceptor outer segments in an age-dependent manner, then develop RPE and photoreceptor degeneration with subretinal neovascularization and sub-RPE wide-spaced collagen deposits. Cp/Heph DKO mice develop retinal, brain, liver, and heart iron overload with iron deficiency anemia. In untreated DKOs, iron gets trapped in the tissues and is not returned to the blood, leading to iron deficiency anemia. There is an age-dependent tissue iron buildup with retinal and brain degeneration.

### Methods

Eye Drop Administration: Beginning at age 5 months (when elevated retinal iron levels are detected), mice were given deferiprone eye drops (10 mg/ml) three times a day in one eye and control water eye drops in the other eye for two months, with the last drop given 2 h prior to sacrifice. Eyes were fixed in 2% paraformaldehyde/2% glutaraldehyde then sectioned for analysis of morphology (following staining with Toluidine Blue) and iron content (following staining with Perls' Prussian Blue). The number and length of retinal pigment epithelial (RPE) cell hypertrophy and photoreceptor atrophy areas were quantified in deferiprone-treated vs. water-treated control eyes. The intensity of Perls' stain in the ciliary body, retina and RPE were assessed in digital photomicrographs quantifying pixel density.

Oral Administration: DKO mice were given deferiprone in their drinking water at a concentration of (1 mg/ml). The mice typically drink 5 ml of water a day and weigh 30 g. Mice began drinking deferiprone/water at age 7 months since that is the time such mice normally develop the retinal changes consistent with AMD. The animals were sacrificed at various time points that are known to represent significant retinal and neurological damage in untreated animals and the eyes of sacrificed animals were analyzed as noted above.

### Example 1

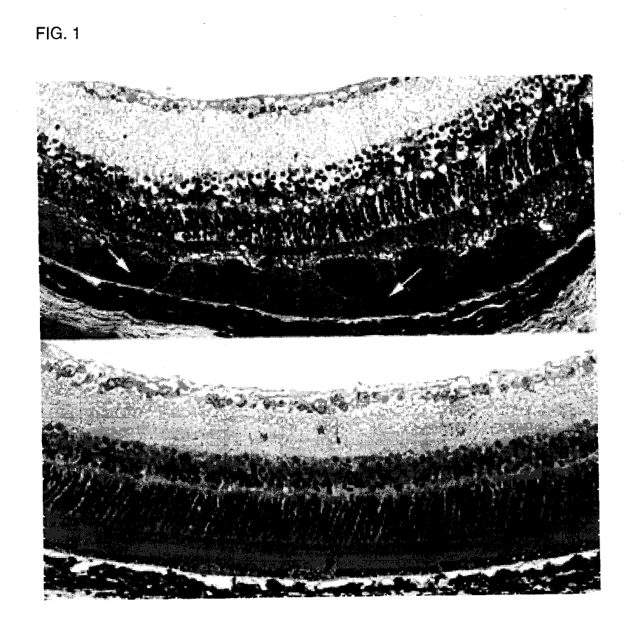

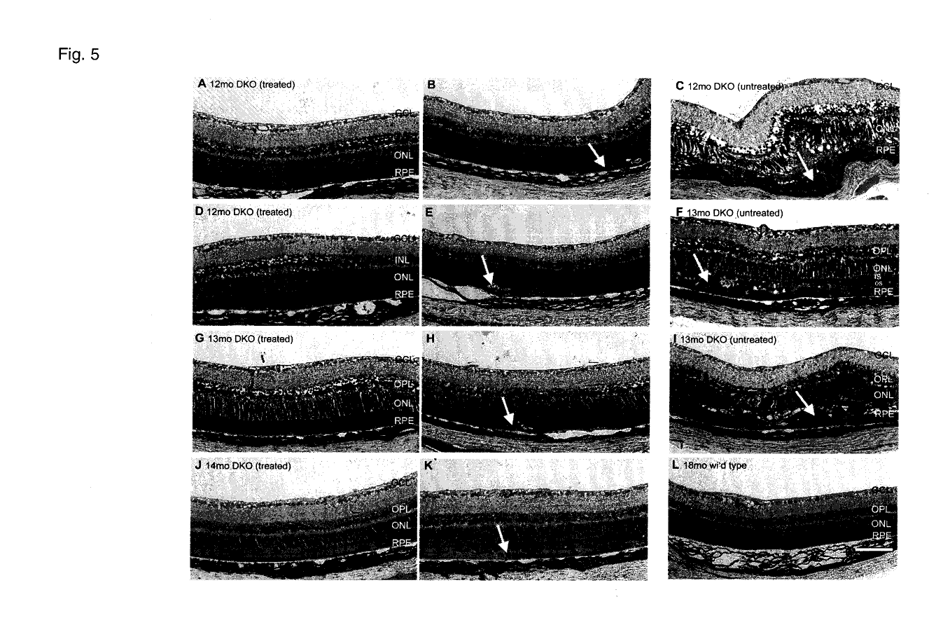

Among 10 DKO mice treated with deferiprone in drinking water at 1 mg/ml, 2 mice lived to 14 months and 2 to 12 months before developing some mild ataxia. Most of the other deferiprone-treated mice were sacrificed for histologic analysis at younger ages, none of which exhibited ataxia. The 12 and 14 month old mice had been on deferiprone for 5-7 months before sacrifice. At the time of sacrifice, their hematocrits were higher than those of untreated mice (30-50% for treated mice compared to 19-26% in untreated controls), suggesting that deferiprone may facilitate transfer to iron to hematopoietic cells. Further, the treated 12-14 month old mice had almost no retinal degeneration and damage. In contrast, retinal damage in the untreated animals was significant at all time points after 7 months and in the few untreated mice that survived to 12-13 months old, all had severe retinal degeneration and damage. (See FIGS. 1 and 5).

Almost all RPE cells in a retina from an untreated, Cp/Heph knockout mouse, age 13 months are abnormal and markedly enlarged (see white arrows in upper panel of FIG. 1), the photoreceptor layer above the RPE cells is thinned, and inner/outer segments are disorganized and degenerated. In contrast, the retina from 14 month Cp/Heph knockout treated with oral deferiprone for 5 months appears normal except for a few mildly hypertrophic RPE cells (see black arrow of lower panel of FIG. 1) and the photoreceptors are normal.

### Example 2

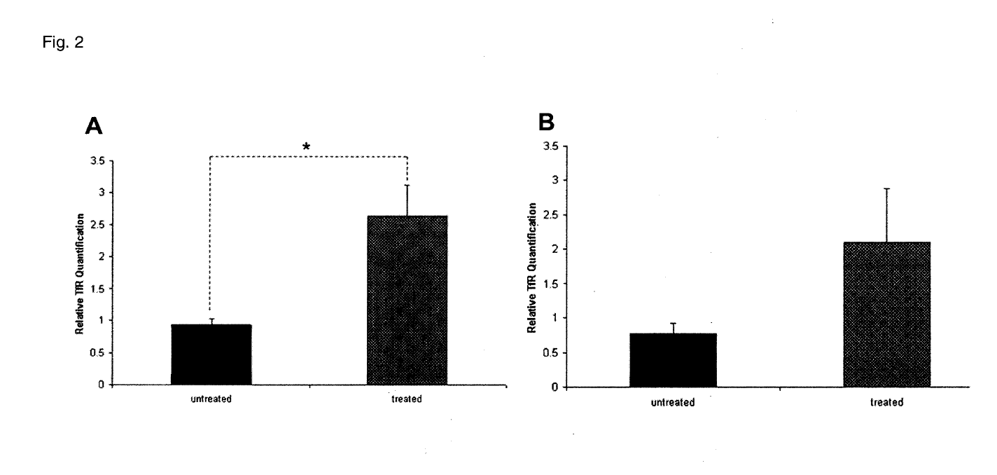

The treated mice (n=4) were given deferiprone for 11 days PO (1 mg/ml in the drinking water). Relative quantification of TfR mRNA, determined by qPCR and standardized to 18S RNA was shown in the retinas (See FIG. 2A) of treated versus untreated mice (n=4), with significant up regulation of TfR mRNA levels shown in the treated animals. Relative quantification of TfR expression is also shown for the RPE/choroids (See FIG. 2B) of treated in comparison to untreated animals. The results are depicted in FIG. 1 and show a significant difference (P<0.05).

### Example 3

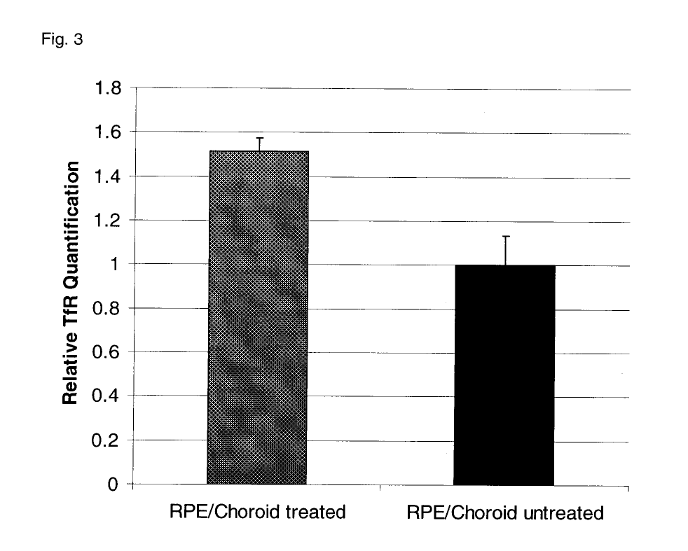

The mouse had the right eye (OD) treated with deferiprone topically (in the form of the eye drops, 10 mg/ml) three times a day for two months, whereas the left eye (OS) was used as an internal control. The last administration was given 2 h prior to sacrifice. Relative quantification of TfR mRNA was detected by qPCR. In the RPE/choroid there is a siqnificant TfR mRNA increase, indicating that deferiprone decreased the labile iron level (See FIG. 3), confirming that the drug will work when administered topically as well. The error bars represent SD of 3 PCR reactions using the same batch of mRNA template.

### Example 4

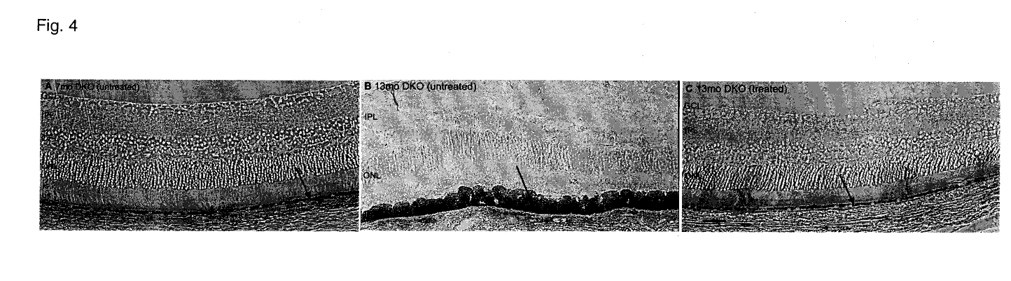

Deferiprone treated Cp-I-Heph-I- (DKO) mice had decreased retinal iron in comparison to untreated DKO mice. Seven and 13 month old untreated DKO mice retinas (see FIGS. 4A and 4B) have more detectable Perls' stain (arrows) in comparison to 13 month old DKO mouse (See FIG. 4C) treated with deferiprone PO, 1 mg/ml in drinking water for 9 months (arrow).

### Example 5

DKO mice have an age-dependent retinal degeneration. Untreated 12 and 13 month old animals (See FIGS. 5C, 5F and 5I), have massive areas of RPE hypertrophy involving more then 90% of the retina (arrows), focal photoreceptor degeneration consisting of thinning of the ONL, inner segment vacuolization, and loss of outer segments. In contrast, age-matched and older DKO mice (See FIGS. 5A, 5D, 5G and 5J) that received deferiprone 1 mg/ml PO in the drinking water for 6-9 months, had near normal tissue. Examination of these retinas for the most severely affected regions in the treated mice revealed only small focal areas of RPE hypertrophy (See FIGS. 5B, 5E, 5H and 5K) involving less then 10% of the retina with otherwise normal appearing retinas. The mice depicted in FIGS. 5A and 5D were treated for 7 months, 5G for 9 months, and 5J for 6 months.

### Example 6

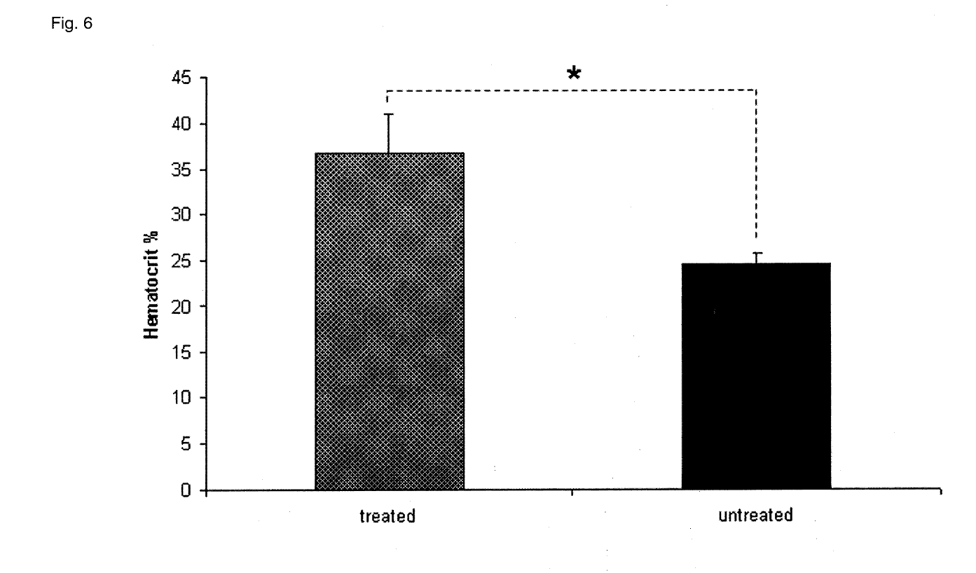

Relative to untreated DKO mice (n=7) which have abnormally low hematocrit values (mean value 24.6%), deferiprone-treated DKO mice (n=7) show significantly higher values (mean value 36.7%) The results are depicted in FIG. 6 and show a significant difference (P<0.05).

### Example 7

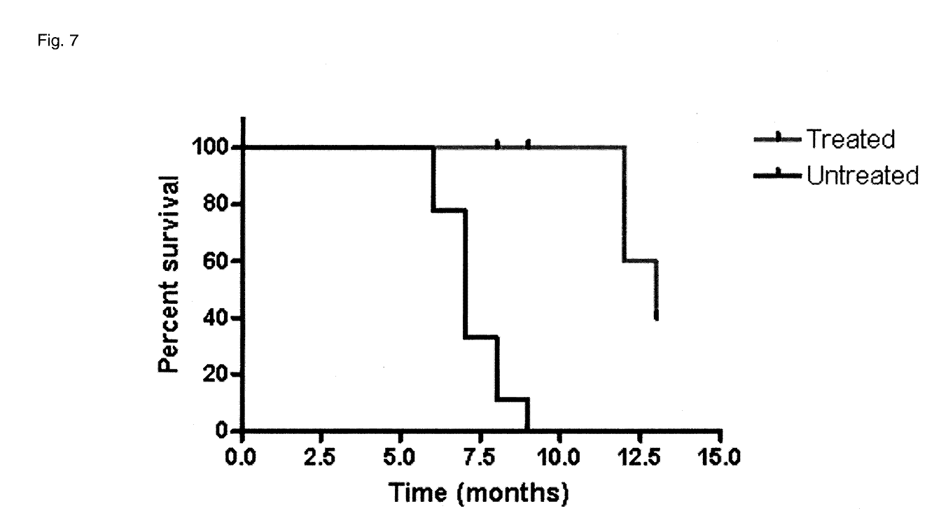

In comparison with untreated DKO animals with median survival of 7 months (expressed by LogRank test), treated animals have significantly extended lifespan (median survival 13 months). (P<0.0001). The lifespan of the treated animals is an under-estimate, as these mice were sacrificed for iron quantification before they became sick. The results are depicted in FIG. 7.

### Example 8

Sensitivity Testing of Eye Drops: To insure the deferiprone solution would not be irritating or damaging to the eye itself, experiments were conducted in rabbits, an animal known to exhibit a high degree of sensitivity to xenobiotics, especially when administered to the eye. The ocular irritation/corrosion potential of the product was assessed by instilling approximately 0.1 mL at a concentration of about 13 mg/mL into the everted lower lid of the right eye of 3 male Hra:(NZW)SPF rabbits; the left eye served as the untreated control. Approximately 24 hours after instillation, the treated eyes were examined for corneal injury and washed using room-temperature physiological saline. Eye irritation was evaluated and scored using the Draize technique at approximately 1, 24, 48, and 72 hours after instillation; no evidence of ocular irritation or corrosion (Score of 0) was noted at any of these observation times. At approximately 24 hours post dose, the sodium fluroscein tests showed no injury to the cornea any of the animals, demonstrating that the formulation was neither irritating nor corrosive and thus suitable for use.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. Furthermore, numeric ranges are provided so that the range of values is recited in addition to the individual values within the recited range being specifically recited in the absence of the range. The word “comprising” is used herein as an open-ended term, substantially equivalent to the phrase “including, but not limited to”, and the word “comprises” has a corresponding meaning. As used herein, the singular forms “a”, “an” and “the” include plural references unless the context clearly dictates otherwise. Thus, for example, reference to “a thing” includes more than one such thing. Citation of references herein is not an admission that such references are prior art to the present invention. Furthermore, material appearing in the background section of the specification is not an admission that such material is prior art to the invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

1-4. (canceled)

5. A method of treatment for an iron-related eye disorder selected from the group consisting of: glaucoma, cataract, diabetic retinopathy, hereditary retinal degeneration, retinal detachment, ischemic retinopathy caused by retinal vein or artery occlusions, ischemic optic neuropathy, optic neuritis, and traumatic optic neuropathy, the method comprising administering a therapeutically effective amount of deferiprone to the eye of a subject having an iron-related eye disorder.

6. The method of claim 5 wherein the deferiprone is administered orally.

7. The method of claim 5 wherein the deferiprone is administered topically.

8-13. (canceled)

14. A method of treatment for iron-related eye disorders, the method comprising topically administering a therapeutically effective amount of deferiprone to theeye of a subject having an iron-related eye disorder.

15. The method of claim 14 wherein the iron-related eye disorder is selected from the group consisting of age-related macular degeneration, glaucoma, cataract, diabetic retinopathy, hereditary retinal degeneration, retinal detachment, ischemic retinopathy caused by retinal vein or artery occlusions, ischemic optic neuropathy, optic neuritis, and traumatic optic neuropathy.

16. The method of claim 14 wherein the iron-related eye disorder is age-related macular degeneration.

17. The method of claim 16 wherein the age-related macular degeneration is exudative.

18. The method of claim 16 wherein the age-related macular degeneration is nonexudative.

19. The method of claim 14 wherein the subject has previously been treated for an iron-related eye disorder.

20. The method of claim 16 wherein the subject has previously been treated for age-related macular degeneration.

21-31. (canceled)

32. A method of preventing iron-induced damage to an eye of a subject at risk for iron-induced eye damage, the method comprising administering a prophylactically effective amount of deferiprone to the subject.

33. The method of claim 32 wherein the eye damage is a physical distortion of the retina.

34. The method of claim 33 wherein the physical distortion of the retina is scarring.

35. The method of claim 32 wherein the eye damage is abnormal vascular growth.

36. The method of claim 32 wherein the iron-induced eye damageis associated with an iron-related eye disorder is selected from the group consisting of age-related macular degeneration, glaucoma, cataract, diabetic retinopathy, hereditary retinal degeneration, retinal detachment, ischemic retinopathy caused by retinal vein or artery occlusions, ischemic optic neuropathy, optic neuritis, and traumatic optic neuropathy.

37. The method of claim 32 wherein the iron-induced eye damage is associated with age-related macular degeneration.

38. The method of claim 37 wherein the age-related macular degeneration is exudative.

39. The method of claim 37 wherein the age-related macular degeneration is nonexudative.

40. The method of claim 32 wherein the subject has previously been treated for eye damage associated with iron.

41. The method of claim 37 wherein the subject has previously been treated for age-related macular degeneration.

42. The method of claim 32 wherein the deferiprone is administered orally.

43. The method of claim 32 wherein the deferiprone is administered topically.
